(19) United States
(12) Patent Application Publication
(10) Pub. No.: US 2007/0010862 A1
(43) Pub. Date: Jan. 11, 2007

(54) Apparatus and method for providing an electrical field to a targeted area

(76) Inventors: Thomas Osypka, Palm Harbor, FL (US); Hyound-Ihl Kim, Palm Harbor, FL (US)

Correspondence Address: EDWARDS & ANGELL, LLP, P.O. BOX 55874, BOSTON, MA, 02205, US

(21) Appl. No.: 11/317,165

(22) Filed: Dec. 23, 2005

Related U.S. Application Data
(60) Provisional application No. 60/674,874, filed on Apr. 26, 2005.

(51) Int. Cl. A61N 1/00

(52) U.S. Cl. 607/115

(57) ABSTRACT

An electrical stimulation apparatus for providing a unipolar electrical field over a targeted area, which includes an elongated flexible lead body having opposed proximal and distal end portions, the proximal end portion of the lead body including a connector for operative connection with a pulse generator, and an electrode assembly connected with the distal end portion of the lead body and electrically connected to the connector. The electrode assembly includes a paddle member having defining a plurality of corner sections. Suture sites located on the paddle member for anchoring the paddle member at an implantation site adjacent a targeted area, and a plurality of electrodes supported on the paddle member and connected to the lead body, wherein each corner section of the paddle member supports a respective one of the plurality of electrodes so as to define an electrode array for generating a unipolar electrical field at the targeted area.

### CROSS-REFERENCE TO RELATED APPLICATION

This application claims the benefit of priority to U.S. Provisional Patent Application No. 60/674,874, filed Apr. 26, 2005, which is incorporated herein by reference.

### BACKGROUND OF THE INVENTION

1. Field of the Invention

The subject technology pertains to an electrical stimulation device that includes a stimulation apparatus used to generate an even electrical field on a targeted area, and more particularly, to a stimulation apparatus that is adpated and condigured to be implanted in the brain area to provide long-term stimulation without migration.

2. Background of the Related Art

The effect of electrical stimulation on the central nervous system (CNS) has been investigated for many years. Early on, electrical stimulation was used to map specific functional areas of the brain, such as motor and language. However, electrical stimulation is currently achieving more success for therapeutic purposes. For example, electrical stimulation is known to improve neural connectivity and help restore function from movement disorders, epilepsy, intractable psychiatric disorders, stroke and coma. Electrical activation or inhibition for CNS disorders is applicable to virtually any brain or spinal cord dysfunction. Accordingly, many devices have been developed to meet the necessity of these types of treatment. However, when stimulation assemblies, and particularly paddle shaped electrodes, are implanted in the brain area, the connecting lead associated therwith typically extends posteriorly from the cranial opening, behind the ear to the anterior chest. Consequently, the connecting lead can become undesirably kinked or distorted.

Recently, the generation of an electrical field near a targeted area of the brain has been suggested to beneficially treat CNS disorders by other mechanisms. Electrical stimulation can inhibit the “neurogenic inflammation” which develops in the early period of cerebral infarction. Inhibition of the macrophage infiltration may diminish the zone of apoptosis due to cerebral infarction, thus reducing the post-infarction sequelae and improving the quality of life. Unipolar stimulation is known to be better than bipolar stimulation in this situation, since cerebral infarction usually affects a deeper portion of the brain. It would be benficial therefore, to provide a unipolar electical stimulation device having a paddle shaped electrode adpated to be implanted in such a manner so as to provide an evenly distributed unipolar elecrical field to a targeted area located deep within the brain of a patient, which that will overcome the deficiencies of the prior art.

### SUMMARY OF THE INVENTION

In view of the above, there is a need for an electrode that can be easily implanted and provide an even, unipolar field deep into a targeted area.

It is an object to implant an electrode that provides an even electrical field over a wider area of the brain with a unipolar electrical field. It is another object to provide an electrode that does not migrate from the targeted area during long-term stimulation.

It is an object of the subject technology to generate a unipolar electrical field deep inside of the brain for attracting oppositely charged implanted cells. It is still another object to provide an electrode that is easily implanted.

The present invention is directed to an electrical stimulation apparatus for providing a unipolar electrical field over a targeted area. The electrical stimulation apparatus includes an elongated flexible lead body having opposed proximal and distal end portions, the proximal end portion of the lead body including a connector for operative connection with a pulse generator and an electrode assembly operatively associated with the distal end portion of the lead body and electrically connected to the connector. The electrode assembly includes a generally planar paddle member having defining a plurality of corner sections, at least one suture site located on the paddle member for anchoring the paddle member at an implantation site adjacent a targeted area and a plurality of electrodes supported on the paddle member and operatively connected to the lead body, wherein each corner section of the paddle member supports a respective one of the plurality of electrodes so as to define an electrode array for generating a unipolar electrical field over the targeted area.

In another embodiment, the subject technology is directed to an electrical stimulation apparatus for providing a unipolar electrical field over a targeted area of the brain, including an elongated flexible lead body having opposed proximal and distal end portions, a connector operatively associated with the proximal end portion of the lead body for operative connection with an implanted pulse generator and an electrode assembly operatively associated with the distal end portion of the lead body and electrically connected to the connector. The electrode assembly includes a generally polygonal paddle member defining a plurality of corner sections, a suture site located at each corner section for securing the paddle member at an implantation site adjacent a targeted area of the brain and an electrode disposed at each corner section of the paddle member so as to define an electrode array for generating a unipolar electrical field over the targeted area. The electrical stimulation apparatus also includes conductor means extending through the lead body for electrically connecting the electrodes of the electrode assembly to the connector.

In another embodiment, the subject technology is directed to and electrical stimulation apparatus for providing a unipolar electrical field over a targeted area of the brain, including an elongated flexible lead body having opposed proximal and distal end portions, a connector operatively associated with the proximal end portion of the lead body for operative connection with an implanted pulse generator, and an electrode assembly operatively associated with the distal end portion of the lead body and electrically connected to the connector. The electrode assembly includes a generally square paddle member defining four corner sections, a suture aperture located at each corner section for securing the paddle member at an implantation site adjacent a targeted area of the brain and an electrode disposed at each corner section of the paddle member so as to define an electrode array for generating a unipolar electrical field over the targeted area. The electrical stimulation apparatus also includes a conductor means extending through the lead body for electrically connecting the electrodes of the electrode assembly to the connector.

It should be appreciated that the present invention can be implemented and utilized in numerous ways, including without limitation as a process, an apparatus, a system, a device, a method for applications now known and later developed. These and other unique features of the system disclosed herein will become more readily apparent from the following description and the accompanying drawings.

### BRIEF DESCRIPTION OF THE DRAWINGS

So that those having ordinary skill in the art to which the disclosed system appertains will more readily understand how to make and use the same, reference may be had to the drawings as follows.

FIG. 1 shows a partial cross-sectional side view of a patient with a stimulation apparatus implanted in the cranial openings, connected to a pulse generator in accordance with the subject technology.

FIG. 2 shows a top view of polygonal shaped stimulation apparatus of FIG. 1 including a 5-electrode array, apertures in three corners, and a connecting lead.

FIG. 3 shows a side view of stimulation apparatus shown in FIG. 1.

FIG. 4 shows a magnified view of FIG. 3, highlighting the stimulation apparatus fixed with dura.

FIG. 5 shows the generation of electrical field from the surface to a deeper portion of the target brain in accordance with the subject technology.

### DETAILED DESCRIPTION OF PREFERRED EMBODIMENTS

The present invention overcomes many of the prior art problems associated with stimulating brain tissue. The advantages, and other features of the systems and methods disclosed herein, will become more readily apparent to those having ordinary skill in the art from the following detailed description of certain preferred embodiments taken in conjunction with the drawings which set forth representative embodiments of the present invention and wherein like reference numerals identify similar structural elements.

In breif overview, a stimulation apparatus is provided for treating CNS disorders, for which generation of an electrical field can be applied. Generation of an electrical field can be an important mechanism to restore the lost function of CNS. Some examplary situations for beneficial use of this electrical field generation include, but are not limited to, the acute destructive disorders where inhibition of “neurogenic inflammation” is required; chronic destructive disorders where implantation of stem cell is required; and some disorders where neuromodulation is required. Further, an electrical field may be applied to CNS damage from origins as varied as congenital, traumatic, degenerative, coma, psychiatric disorders such as depression and epilepsy, central pain syndromes, stroke and the like.

The stimulation apparatus can be implanted epidurally and subdurally. For example, a craniotomy may be required to subdurally implant the stimulation apparatus. Location is dependent on the nature of the disease and the treatment desired. For instance in alleviating central pain, the stimulation apparatus is implanted over the motor cortex. For neocortical epilepsy, the stimulation apparatus is implanted over the presumed epeileptogenic area. For damaged brain, the stimulation apparatus is implanted over the surrounding compensatory area.

### The Stimulation Apparatus

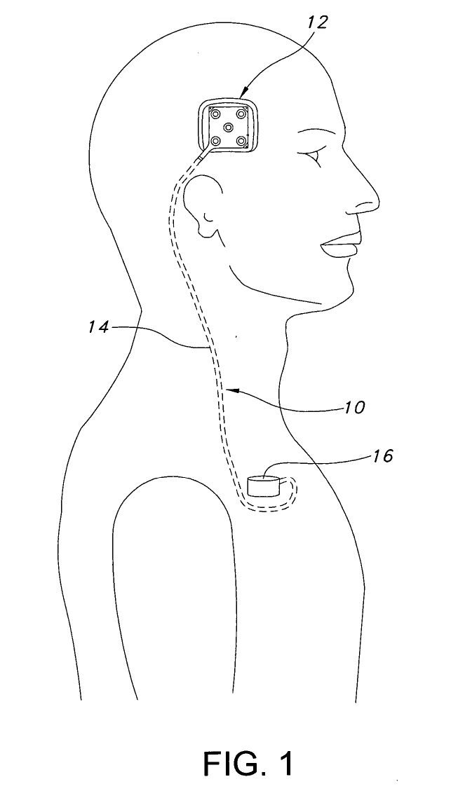

Referring now to FIG. 1, a stimulation apparatus is designed for generating an electrical field in a targeted area of the brain and referred to generally by the reference numeral 10. The stimulation apparatus 10 includes an electrode assembly 12 for implantation in the target area. A connecting lead 14 passes from a corner of the electrode assembly 12 and provides power and passes necessary electrical signals from a pulse generator 16 to the electrode assembly 12. As such, the pulse generator 16 can be implanted in a plurality of locations, preferably the chest area.

The electrode assembly 12 has a polygonal configuration as to be easily adjusted along the curve from a cranial opening to the location of the pulse generator 16, usually the anterior chest. The generally planar, polygonal shape (square as shown) is designed such that the electrode assembly 12 is easily inserted inside a cranial opening. The connecting lead 14 is blended into a corner to prevent kinking. Further, the polygonal shape yields improved coverage of the functional compartment of the brain, as most functional compartments are divided by gyrus and sulcus running horizontally or vertically. By trailing the lead 14 to a corner of the electrode assembly 12, the surgeon has flexibility with the lead 14 to move to a desired direction with less strain while the electrode assembly 12 is adjusted to the shape of each functional compartment.

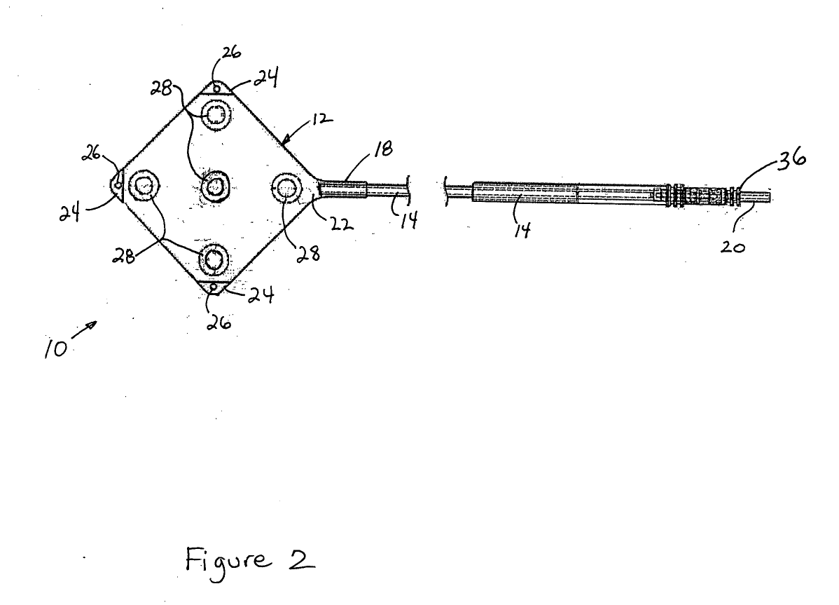

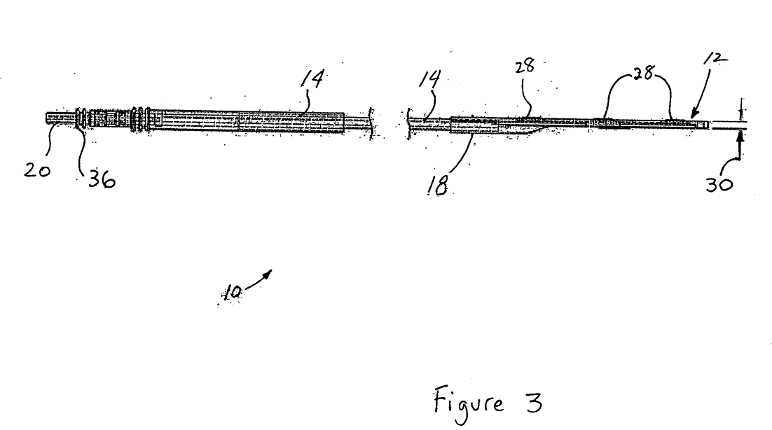

Referring now to FIGS. 2 and 3, top and side detailed views, respectively, of the stimulation apparatus 10 are shown. The connecting lead 14 has a distal end 18 adapted for connection to the electrode assembly 12 and a proximal end 20 having a standard connector 36 adapted for connection to the pulse generator 16. The electrode assembly 12 has a first corner 22 connected to the distal end 18 of the lead. The electrode assembly 12 is blended into the connecting lead 14 with consideration of the durable resistance against an unexpected force. The electrode assembly 12 has three additional corners 24 for anchoring the electrode assembly 12 in place. Each corner 24 has a suture site such as a hook or fabric that can be pierced. Preferably, each corner 24 defines an aperture 26 sized and configured for epidural fixation. Thus, after implanting the electrode assembly 12, migration is prevented during long-term stimulation.

Five electrodes 28 mount on the electrode assembly 12 and operatively connect to the lead 14. Four electrodes 28 of the five electrodes 28 are mounted near the corners 22, 24, respectively, and the fifth electrode 28 is substantially centrally mounted on the electrode assembly 12. The fifth, cental electrode 28 is positioned to supplement a “blank” area of the electrical field in order that an even electrical field is acheived. Each electrode 28 is connected to the other with wires (not shown). Thus, when the electrodes 28 receive an electrical signal from the pulse generator 16, the electrodes 28 generate a positive or a negative electric field. In one embodiment, each electrode 28 is an annular ring electrode.

It is envisioned that various polygons may form a plurality of corners and central locations that may receive electrodes and suture sites. More or fewer electrodes 28 could be utilized in a variety of arrangements. For example, the number of electrodes 28 may be increased and/or electrodes 28 may be utilized intermediate the corners and centrally to achieve a stronger or more even electrical field. Additionally, the size of the electrode assembly 12 may be increased or decreased to suit a particular application. For example, if the size of the electrode assembly 12 is increased, a concomitant increase of electrodes 28 can be considered with a similar configuration. The electrode assembly may be a polygon of three or more sides, which may be regular or irregular in configuration. The polygon may also include one or more portions that are not linear. As a result, the various polygons can be used to facilitate implantation and tailor the shape of the field generated thereby. In one embodiment configured as shown in the figures, the width of the paddle is about 2 cm and a thickness 30 of the paddle is about 1.15 cm. The diameter of the fixing apertures 26 is about 0.25 cm. Preferably, but without limitation, the diameter of the electrode varies between 2 mm to 8 mm, the width of the paddle varies from 2 cm to 4 cm, the thickness varies from about 0.75 mm to 1.5 cm and the diameter of the fixing apertures varies from 0.25 mm to 1.5 mm although any desirable configuration could be a achieved by one of ordinary skill in the pertinent art.

### Implantation and Use of the Stimulation Apparatus

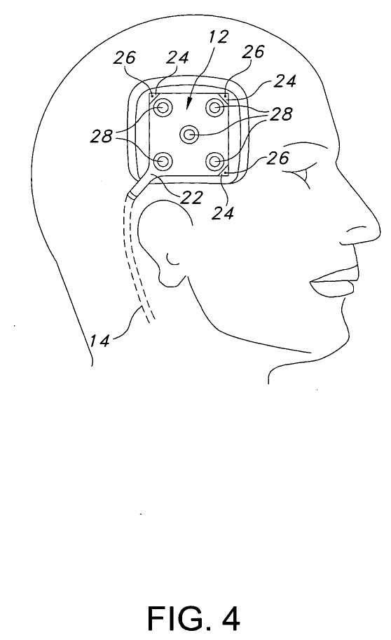

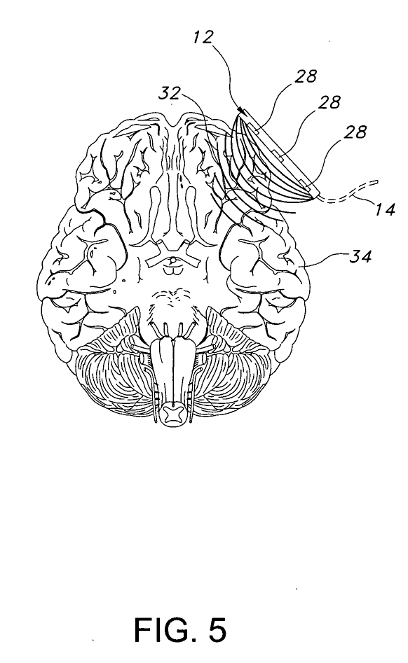

Referring now to FIGS. 4 and 5, the stimulation apparatus 10 is shown implanted and in use, respectively. The stimulation apparatus 10 may be used with any kind of implantable pulse generator and the pulse generator 16 is preferably located in the anterior chest. To implant the electrode assembly 12, a target site of the brain is determined according to the extent of the damage in the target area of the brain, the purpose of the treatment, and the mechanism by which a lost brain function may be restored. Exemplary situations are: for chronic central damage, when the electrode assembly 12 is usually located in the motor cortex; for chronic intractable neocortical epilepsy, where the epileptogenic area must be figured out by other methods such as magnetic resonance imaging and/or invasive recording; for damaged brain to be treated with stem cells when the electrode assembly 12 is located over the center of damage where stem cells are collected; and for neurogenic inflammation when the electrode assembly 12 is placed in the periphery of the lesion.

Once the target is determined, a patient is anesthetized, locally or generally. A surgeon cuts about a 6×6 cm portion of scalp and reflects the cut portion down. One burr hole is made in the postero-inferior point of the presumed craniotomy. Bone flap is made in a square fashion using a bone saw. The electrode assembly 12 is place epidurally or subdurally over the target sites of the brain. The electrode assembly 12 is fixed to dura with silk using the fixing apertures 26. Bone flap is replaced and fixed with the connecting lead 14 passing through the burr hole without any strain. The connecting lead 14 is passed subcutaneously to the anterior chest and connected to the pulse generator 16. As shown in FIG. 5, upon subsequent pulses being sent from the pulse generator 16 to the electrode assembly 12, an electrical field 32 is generated within the brain 34.

Stem cell therapy is increasingly gaining attention for the treatment of CNS disorders. Stem cells are implanted locally in the lesioned area or introduced in the veins. However, one of the challenges to delivering stem cells is that a number of cells are not easily settled in a target lesion area. To facilitate the settlement, the subject electrode 10 can be used to generate a positively-charged electrical field over the targeted area. The positively-charged electrical field helps to collect and stabilize electrically charged stem cells thereto resulting in improved settlement.

The aforedescribed electrode can be of two types, unipolar or bipolar as would be known to those of ordinary skill in the pertinent art. As is known in the bipolar type, two electrodes are present and a pulse is applied between them. In the unipolar type, each electrode and the body saline solution and/or body tissue, which are electrically conductive, provide the return electric path between the electrode and an outer plate, which is electrically connected to the associated circuitry. One example of a prior art unipolar electrode is described in U.S. Pat. No. 3,735,766, which is incorporated herein by reference.

Unless otherwise specified, the illustrated embodiments can be understood as providing exemplary features of varying detail of certain embodiments, and therefore, unless otherwise specified, features, components, modules, elements, and/or aspects of the illustrations can be otherwise combined, interconnected, sequenced, separated, interchanged, positioned, and/or rearranged without materially departing from the disclosed systems or methods. Additionally, the shapes and sizes of components are also exemplary and unless otherwise specified, can be altered without materially affecting or limiting the disclosed technology.

While the invention has been described with respect to preferred embodiments, those skilled in the art will readily appreciate that various changes and/or modifications can be made to the invention without departing from the spirit or scope of the invention as defined by the appended claims.

What is claimed is:

1. Electrical stimulation apparatus for providing a unipolar electrical field over a targeted area comprising:
a) an elongated flexible lead body having opposed proximal and distal end portions, the proximal end portion of the lead body including a connector for operative connection with a pulse generator; and
b) an electrode assembly operatively associated with the distal end portion of the lead body and electrically connected to the connector, the electrode assembly including:
i) a generally planar paddle member having defining a plurality of corner sections;
ii) at least one suture site located on the paddle member for anchoring the paddle member at an implantation site adjacent a targeted area; and
iii) a plurality of electrodes supported on the paddle member and operatively connected to the lead body, wherein each corner section of the paddle member supports a respective one of the plurality of electrodes so as to define an electrode array for generating a unipolar electrical field over the targeted area.

2. The electrical stimulation apparatus of claim 1, further comprising a central electrode supported on the paddle member equidistant from each of the corner supported electrodes.

3. The electrical stimulation apparatus of claim 1, further comprising a pulse generator operatively associated with the connector at to the proximal end of the lead body.

4. The electrical stimulation apparatus of claim 1, further comprising electrical conducting means extending through the lead body form electrically connecting the electrodes of the electrode assembly with the connector at the proximal end pf the lead body.

5. The electrical stimulation apparatus of claim 1, wherein the paddle member has a generally polygonal shape.

6. The electrical stimulation apparatus of claim 1, wherein a suture site is associated with each corner section of the paddle member.

7. The electrical stimulation apparatus of claim 1, wherein a suture site is defined at least in part by an aperture.

8. The electrical stimulation apparatus of claim 1, wherein each electrode of the electrode assembly is defined by an annular ring electrode.

9. The electrical stimulation apparatus of claim 1, wherein the distal end portion of the lead body is operatively associated with a corner section of the paddle member.

10. Electrical stimulation apparatus for providing a unipolar electrical field over a targeted area of the brain, comprising:
a) an elongated flexible lead body having opposed proximal and distal end portions;
b) a connector operatively associated with the proximal end portion of the lead body for operative connection with an implanted pulse generator;
c) an electrode assembly operatively associated with the distal end portion of the lead body and electrically connected to the connector, the electrode assembly including:
i) a generally polygonal paddle member defining a plurality of corner sections;
ii) a suture site located at each corner section for securing the paddle member at an implantation site adjacent a targeted area of the brain; and
iii) an electrode disposed at each corner section of the paddle member so as to define an electrode array for generating a unipolar electrical field over the targeted area; and
d) conductor means extending through the lead body for electrically connecting the electrodes of the electrode assembly to the connector.

11. The electrical stimulation apparatus of claim 10, further comprising a central electrode supported on the paddle member equidistant from each of the corner supported electrodes.

12. The electrical stimulation apparatus of claim 11, wherein the paddle member has a generally square configuration defining four corner sections.

13. The electrical stimulation apparatus of claim 12, wherein the suture site located at each corner section of the paddle member is spaced outwardly from the electrode disposed at such corner section of the paddle member.

14. The electrical stimulation apparatus of claim 10, wherein each suture site is defined at least in part by an aperture.

15. The electrical stimulation apparatus of claim 10, wherein each electrode of the electrode assembly is defined by an annular ring electrode.

16. The electrical stimulation apparatus of claim 10, wherein the distal end portion of the lead body is operatively associated with a corner section of the paddle member.

17. Electrical stimulation apparatus for providing a unipolar electrical field over a targeted area of the brain, comprising:
a) an elongated flexible lead body having opposed proximal and distal end portions;
b) a connector operatively associated with the proximal end portion of the lead body for operative connection with an implanted pulse generator;
c) an electrode assembly operatively associated with the distal end portion of the lead body and electrically connected to the connector, the electrode assembly including:
i) a generally square paddle member defining four corner sections;
ii) a suture aperture located at each corner section for securing the paddle member at an implantation site adjacent a targeted area of the brain; and
iii) an electrode disposed at each corner section of the paddle member so as to define an electrode array for generating a unipolar electrical field over the targeted area; and
d) conductor means extending through the lead body for electrically connecting the electrodes of the electrode assembly to the connector.

18. The electrical stimulation apparatus of claim 17, further comprising a central electrode supported on the paddle member equidistant from each of the corner supported electrodes.

19. The electrical stimulation apparatus of claim 17, wherein each electrode of the electrode assembly is defined by an annular ring electrode and the distal end portion of the lead body is operatively associated with a corner section of the paddle member.

20. A method for settling stem cells as therapy comprising the steps of:
implanting stem cells locally in a lesioned area;
facilitating settlement of the stem cells by generating a positively-charged electrical field over the targeted area such that the positively-charged electrical field helps to collect and stabilize electrically charged stem cells thereto.

21. A method as recited in claim 20, further comprising the step of charging the stem cells.
